(19) United States
(12) Patent Application Publication
(10) Pub. No.: US 2026/0151347 A1
(43) Pub. Date: Jun. 4, 2026

(54) CELL-DERIVED MICROPARTICLE DELIVERY SYSTEM AND USES THEREOF

(71) Applicants: Jason P. Gleghorn, Lincoln University, PA (US); Michael Donzanti, Newark, DE (US); Ryan Zurakowski, Newark, DE (US)

(72) Inventors: Jason P. Gleghorn, Lincoln University, PA (US); Michael Donzanti, Newark, DE (US); Ryan Zurakowski, Newark, DE (US)

(73) Assignee: University of Delaware, Newark, DE (US)

(21) Appl. No.: 18/707,416

(22) PCT Filed: Nov. 2, 2022
(86) PCT No.: PCT/US2022/048686

§ 371 Date: May 3, 2024

Related U.S. Application Data
(60) Provisional application No. 63/275,027, filed on Nov. 3, 2021.

(51) Int. Cl. A61K 9/50; A61K 9/00; A61K 39/00

(52) U.S. Cl. CPC A61K 9/5068; A61K 9/0085; A61K 9/5052; A61K 9/5089; A61K 39/00

(57) ABSTRACT

The invention relates to a microparticle comprising a core and a membrane surrounding the core, wherein the membrane comprises a cell membrane component. A method for transporting the microparticle is provided. The transport method comprises administering the microparticle to an endothelium, whereby the microparticle is bound to the endothelium; and moving the microparticle across the endothelium. Also provided is a method for preparing the microparticle.

### CROSS-REFERENCE TO RELATED APPLICATION

This application claims priority to U.S. Provisional Application No. 63/275,027, filed Nov. 3, 2021, and the contents of which are incorporated herein by reference in their entireties for all purposes.

### FIELD OF THE INVENTION

This invention relates generally to cell-derived microparticles useful as a delivery system for crossing an endothelium barrier and uses and preparation thereof.

### BACKGROUND OF THE INVENTION

Targeted drug delivery to many parts of the body remains a central challenge, particularly into the central nervous system (CNS) and lymph node (LN) due to a selective and restrictive endothelium. The endothelium is a tissue that separates circulating blood and lymph fluid from the tissues in the body. As such, all fluid, molecules, macromolecules, and cells that move from the circulating bloodstream or lymph fluid must cross endothelial barriers. Dysregulated vascular endothelium that occurs in tumors and other pathological growth allow the passive transport of fluid, molecules and nanoscale sized aggregates (or nanoparticles), and cells. However, in the absence of pathology, normal endothelium acts as a selective barrier with regional barrier properties in different tissues and in different types of blood vessels. For instance, in some parts of the body the endothelium is naturally “leaky” with large fenestrations such as in the bone marrow or capillary networks in tissues such as muscle. This architecture enables passive transport of fluid, molecules, and nanoscale sized aggregates (or nanoparticles) and active squeezing of cells through open capillary fenestrations as documented with podocyte formation and active processes from circulating cells. Other sites in the body, including the blood-brain barrier (BBB) in the central nervous system (CNS) and the high endothelial venule (HEV) in the lymph node (LN), have similar characteristics that severely restrict the passive transport of most small molecule drugs into these sites. Conventionally, the dogma is that circulating cells can transit these endothelial barriers by actively extravasating through the endothelium, classically depicted in the literature as a cell squeezing through the endothelium. Several known mechanisms do exist for trans-endothelial cellular transport; however, descriptions of these mechanisms rely on active processes from the extravasating cell.

There remains a need for an effective delivery system to transport various active agents across endothelial barriers to target sites in, for example, brain and lymph nodes.

### SUMMARY OF THE INVENTION

The inventors have surprisingly discovered non-naturally occurring microparticles capable of crossing an endothelium. The present invention relates to the microparticles for delivering active agents across an endothelial barrier to target sites in, for example, brain and lymph nodes.

A microparticle is provided. The microparticle comprises a core and a membrane surrounding the core, and the membrane comprises a cell membrane component.

The membrane may further comprise a synthetic membrane component.

The membrane may be from a permeabilized cell. The permeabilized cell may have been subject to cryopermeabilization, a detergent, or a chemical permeabilization solution. The permeabilized cell may be a permeabilized leukocyte.

The membrane may further comprise a targeting moiety. The targeting moiety may comprise an integrin, selectin, cadherin, immunoglobulin-like adhesion molecule, addressin, chemokine receptor, chemokine ligand, growth factor receptor, immunoglobulin superfamily protein, ion channel-linked receptor, G protein-coupled receptor, enzyme-linked receptor, antibody or a fragment thereof, or a binding domain thereof.

The core may comprise cytoplasm, a liquid, a polymer, an extracellular matrix protein, or a combination thereof. The core may comprise an active agent. The active agent may comprise a biological molecule, a chemical compound, or a combination thereof. The active agent may comprise a nanoparticle, a liposome, a virus, or a combination thereof. The active agent may comprise a therapeutic, an imaging agent, a sequestering agent, a prophylactic agent, a diagnostic agent, a prognostic agent, an excipient or a combination thereof. The core may be prepared from a leukocyte.

The microparticle may not be immunogenic.

A method for transporting a microparticle is provided. The microparticle comprises a core and a membrane surrounding the core, and the membrane comprises a cell membrane component. The transport method comprises administering the microparticle to an endothelium, whereby the microparticle is bound to the endothelium; and moving the microparticle across the endothelium. The endothelium may be in brain or a lymph node. The endothelium may be in a subject.

According to the transport method, the membrane may further comprise a targeting moiety. The targeting moiety may comprise an integrin, selectin, cadherin, immunoglobulin-like adhesion molecule, addressin, chemokine receptor, chemokine ligand, growth factor receptor, immunoglobulin superfamily protein, ion channel-linked receptor, G protein-coupled receptor, enzyme-linked receptor, antibody or a fragment thereof, or a binding domain thereof.

The transport method may further comprise moving the microparticle to a target site after moving the microparticle across the endothelium. The endothelium may be in a lymph node and the target site may be a lobule in the lymph node. The endothelium may be in a brain and the target site may be in brain parenchyma or cerebrospinal fluid (CSF). The endothelium and the target site may be in a tumor.

The core may comprise an active agent, and the transport method may further comprise releasing the active agent at the target site.

The transport method may further comprise sequestering a molecule by the microparticle from the target site.

The transport method may further comprise causing a biological response at the target site. The biological response may be selected from the group consisting of immune interactions, cancer therapy, vaccine responses, and immunotherapy.

A method for preparing a microparticle is further provided. The preparation method comprises mixing a core with a membrane, and the membrane comprises a cell membrane component. The membrane may further comprise a synthetic membrane component.

The membrane may be a cell membrane of a permeabilized leukocyte, and the preparation method may further comprise adding the core into the permeabilized leukocyte. The permeabilized leukocyte may be a permeabilized lymphocyte.

The preparation may further comprise wrapping the core with the membrane. The membrane may be a cell membrane isolated from a permeabilized leukocyte, for example. The permeabilized leukocyte may have been subject to cryopermeabilization, a detergent, or a chemical permeabilization solution. The permeabilized leukocyte may be a permeabilized lymphocyte.

According to the preparation method, the membrane may further comprise a targeting moiety. The targeting moiety may comprise an integrin, selectin, cadherin, immunoglobulin-like adhesion molecule, addressin, chemokine receptor, chemokine ligand, growth factor receptor, immunoglobulin superfamily protein, ion channel-linked receptor, G protein-coupled receptor, enzyme-linked receptor, antibody or a fragment thereof, or a binding domain thereof.

The preparation method may further comprise loading the core with an active agent.

The preparation method further comprise preparing the core from a permeabilized leukocyte. The permeabilized leukocyte may have been subjected to cryopermeabilization, a detergent, or a chemical permeabilization solution. The permeabilized leukocyte may be a permeabilized lymphocyte.

### BRIEF DESCRIPTION OF THE DRAWINGS

FIG. 1 shows a schematic representation of how the invention works.

FIG. 2 shows serial block-face SEM images of an HEV cross section shows HEV cell reorganization to allow for lymphocyte transcellular transport.

FIG. 3 shows possible schematic method for production of permeabilized cells as MPs.

FIG. 4 shows live/dead viability staining comparing cell death at different freezing rates and cryoprotectant concentrations. Number in bottom left corner indicated cell death.

FIG. 5 shows no aggregation in a resuspension of MPs without the addition of DNAse (−DNAse), and aggregation with the addition of DNAse (+DNAse).

FIG. 6 shows images of live control Jurkat cells (top panels) and permeabilized CSTL Jurkat cells (MPs) (bottom panels) under brightfield microscopy (left panels), fluorescence microscopy (middle panels) and merged images (right panels).

FIG. 7 shows flow cytometry of (A) live cells, (B) MP pre spin, and (C) MP post spin, illustrating that permeabilizing cells (MPs) cause differences in cell size as compared with live cells.

FIG. 8 shows recovery rate of CSTLs (MPs) under different centrifuge conditions over a range of spin speeds.

FIGS. 9A-B show changes to (A) diameter and (B) circularity following spins at different speeds.

FIGS. 10A-B show changes to (A) diameter and (B) circularity following consecutive spins@300×g for 5 min.

FIGS. 11A-B show changes to (A) diameter and (B) circularity following a two-hour incubation at different temperatures.

FIGS. 12A-D show a microfluidic model of blood flow for stability and vehicle breakdown testing (A). Changes to MP count (B), diameter (C) and circularity (D) after a number of runs through the vessel mimic.

FIGS. 13A-C show (A) release profiles generated with MPs following loading by 70 kDa FITC-Dextran, (B) Raltegravir and (C) Cisplatin, illustrating the wide potential in drug loaded.

FIG. 14 shows images of triple negative breast cancer cells (4T1-luc2) with no treatment (control) or treated in vitro with unloaded MPs, free cisplatin, or cisplatin loaded MPs (cisplatin-MPs) at a dose equivalent that of the free cisplatin.

FIG. 15 shows an image of alginate MPs (Alginate MP) and a cell mimetic membrane-wrapped alginate MP (cmMP), in each of which the core was labelled with Cy5.

FIG. 16 shows an image of an alginate MP, an image of T-cell derived plasma membrane (TcPM), and an image of cell mimetic membrane-wrapped alginate MP (cmMP), in each of which the plasma membrane was labelled with DID for visualization.

FIG. 17 shows an image of alginate MP hydrogel core (Alginate core) labeled with AF647 Gydrazide, an image of T-cell derived plasma membrane (TcPM) labeled by BODIPY TMRC5 Malemide, and a merged image of the Alginate care and the TcPM (cmMP).

FIG. 18 shows a release curve of passively loaded fluoresceinamine in alginate MPs.

FIG. 19 shows a vibratome section of a mouse LN with strong uptake of CFSE labeled MPs in the lobule as compared to a dye only control. Phalloidin counterstain shows clear interaction of MPs and HEV cells, as well as presence outside of the vasculature in the lobule.

FIG. 20 shows diagrams (left panels) and images (right panels) of MP control injection (top panels) and FAB+MP injection validating LN homing capabilities of MPs (bottom panels).

FIG. 21 illustrates an experimental design schematic for brain collection.

FIG. 22A-F show images of control MPs from activated T cells or quiescent T cells in lymph node (A) or brain (D), respectively; targeted MPs from quiescent T cells or activated T cells in lymph node (LN) (B) or brain (E), respectively; and vascular counterstain in lymph node (C) or brain (F). MPs from activated T cells traffic inefficiently to (A) LN but efficiently to brain (E). MPs from quiescent T cells traffic efficiently to the (B) LN but inefficiently to (D) brain. Vascular counterstain confirms extravasation of the MPs into the tissue in both brain and LN (C and E). NHS Cy5.5-labeled MPs administered 4 hours before sacrifice.

FIG. 23 shows 2-Phase Release Kinetics of small molecules from MPs.

FIG. 24 shows a PK/PD model of MP distribution in the body.

FIG. 25 shows predicted concentrations of MPs and small molecule drugs in the plasma and lymph node over time.

### DETAILED DESCRIPTION OF THE INVENTION

The present invention provides cell-derived microparticles (MPs) as a delivery system across an endothelial barrier. The invention is based on the inventors'surprising discovery that, during transport of circulating live cells (e.g., leukocytes) across an endothelium, an endothelial barrier, via extravasation at target tissue sites in, for example, brain and lymph nodes, the endothelial cells in the endothelium shuttle the circulating live cells across the endothelial barrier after the native cells dock to the apical surface of the endothelial cells. In particular, the inventors have unexpectedly discovered that docking of circulating live cells and subsequent trans-endothelial cellular transport (extravasation or diapedesis) are actively regulated by the endothelium while the transport is a passive process for the circulating live cells as defined by the composition of their cell membrane. This is evidenced by the findings that dead T cells are capable of binding to the high endothelial venule (HEV) apical surface and extravasating into a lymph node lobule or entering brain parenchyma. As T cells and other specialized lymphocytes routinely traffic across the endothelial barrier, the inventors have further discovered that cell-mimetic microparticles having a core wrapped with a membrane derived from a cell membrane of cells such as leukocytes (e.g., lymphocytes) are capable of crossing an endothelium. Such a functional property is defined by the composition of the membrane and the size of the core. While nanoparticles are taken up intracellularly and retained within a cell, the microparticles of the present invention are transported across the endothelium.

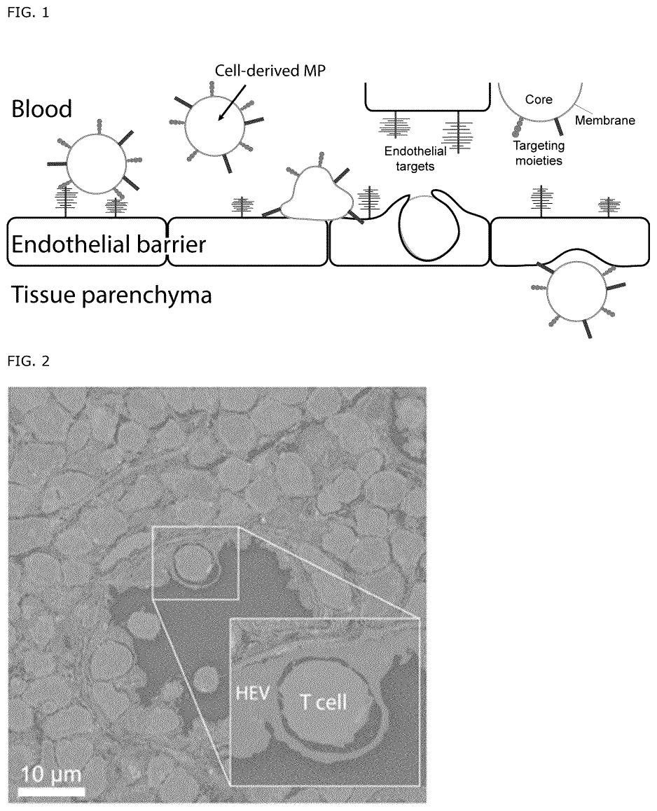

The inventors have developed a MP with a cell-derived membrane to enable docking and interaction with the endothelium and subsequent transport across the endothelium into, for example, tissue parenchyma (FIG. 1). Different membrane compositions, for example, isolated from different cell types, membrane mixtures of cell types, or modified isolated cell membranes, enable the MPs to bind distinct sites on restrictive endothelial barriers in desirable tissues and cross the restrictive endothelial barriers to deliver active agents, also known as payloads (e.g., sequester agents), locally as a drug depot. Additionally, the membrane composition enables the direct interaction with living cells at the target sites to induce a response. These MPs may circulate systemically in the bloodstream or through the lymph fluid in a subject (e.g., human or non-human) to move into tissues throughout the body to locally deliver/sequester agents at target (e.g., therapeutic) sites.

The term “microparticle (MP)” as used herein refers to a substance having a size in the range of about 0.1-1,000 μm, 0.1-900 μm, 0.1-800 μm, 0.1-700 μm, 0.1-600 μm, 0.1-500 μm, 0.1-400 μm, 0.1-300 μm, 0.1-200 μm, 0.1-100 μm, 0.1-50 μm, 0.1-10 μm, 0.1-1 μm, 0.5-1,000 μm, 0.5-900 μm, 0.5-800 μm, 0.5-700 μm, 0.5-600 μm, 0.5-500 μm, 0.5-400 μm, 0.5-300 μm, 0.5-200 μm, 0.5-100 μm, 0.6-1,000 μm, 0.6-900 μm, 0.6-800 μm, 0.6-700 μm, 0.6-600 μm, 0.6-500 μm, 0.6-400 μm, 0.6-300 μm, 0.6-200 μm, 0.6-100 μm, 0.7-1,000 μm, 0.7-900 μm, 0.7-800 μm, 0.7-700 μm, 0.7-600 μm, 0.7-500 μm, 0.7-400 μm, 0.7-300 μm, 0.7-200 μm, 0.7-100 μm, 0.8-1,000 μm, 0.8-900 μm, 0.8-800 μm, 0.8-700 μm, 0.8-600 μm, 0.8-500 μm, 0.8-400 μm, 0.8-300 μm, 0.8-200 μm, 0.8-100 μm, 0.9-1,000 μm, 0.9-900 μm, 0.9-800 μm, 0.9-700 μm, 0.9-600 μm, 0.9-500 μm, 0.9-400 μm, 0.9-300 pm, 0.9-200 μm, 0.9-100 μm, 1-1,000 μm, 1-900 μm, 1-800 μm, 1-700 μm, 1-600 μm, 1-500 μm, 1-400 μm, 1-300 μm, 1-200 μm, 1-100 μm, 100-1,000 μm, 100-900 μm, 100-800 μm, 100-700 μm, 100-600 pm, 100-500 μm, 100-400 μm, 100-300 μm, 100-200 μm, 500-1,000 μm, 500-900 μm, 500-800 μm, 500-700 μm, 500-600 μm, 750-1,000 μm, 750-900 μm or 750-800 μm. For example, the MP may have a size of 0.8-500 μm.

The term “extravasation” as used herein refers to transportation of a microparticle (MP) through a cell barrier.

The terms “cell barrier” and “tissue barrier” are used herein interchangeably and refer to one or more layers of cells that separate two biological spaces in a subject. For example, the cell barrier may be an endothelial barrier.

The terms “endothelial barrier” and “endothelium” are used herein interchangeably and refer to one or more layers of endothelial cells that separate two compartments in a subject. For example, an endothelial barrier may separate a blood vessel from a lymph node lobule.

The term “subject” used herein refers to a mammal, for example, a primate or a human. The subject may be a human or non-human. The subject may have suffered from or be predisposed to a disease or condition.

The term “membrane” as used herein refers to a lipid-based shell comprising a monolayer, bilayer or multilayer. The membrane may comprise a phospholipid bilayer. The membrane may have a thickness of about 0.1-200 nm, 0.1-150 nm, 0.1-100 nm, 0.1-50 nm, 0.1-20 nm, 0.1-10 nm, 0.1-1 nm, 0.5-200 nm, 0.5-150 nm, 0.5-100 nm, 0.5-50 nm, 0.5-20 nm, 0.5-10 nm, 0.5-1 nm, 1-200 nm, 1-150 nm, 1-100 nm, 1-50 nm, 1-20 nm, 1-10 nm, 0.1-1 nm, 5-200 nm, 5-150 nm, 5-100 nm, 5-50 nm, 5-20 nm or 5-10 nm.

The term “cell” as used herein refers to any cell from a subject. The cell may be from a subject that is the same or of the same genus or species of the subject in which a cell barrier is crossed by an MP. The cell may be a blood cell (e.g., red blood cell (RBC), white blood cell (WBC), or platelet). The cell may be an immune cell. The immune cell may be selected from the group consisting of lymphoid progenitor cells and all cells differentiated from that progenitor, including all T cells, B cells, and Natural Killer (NK) cells, NKT cells, Plasma cells, and all subsets and subtypes of these cells. The immune cell may be selected from the group consisting of myeloblast progenitor cells and all cells differentiated from that progenitor cell, including granulocytes (eosinophils, basophils, neutrophils, and mast cells), myeloid-derived suppressor cells, and antigen-presenting cells (APCs), including dendritic cells (plasmacytoid and conventional cell types), monocytes, and macrophages. The immune cell may be selected from innate lymphoid cells, tissue-resident immune cells (e.g., microglial cells), mucosal-associated invariant T (MAIT) cells, and decidual macrophages, decidual natural killer cells. The cell may be of a placental cell. The placental cell may be selected from the group consisting of trophoblasts, placental fibroblasts, and placental endothelial cells, extravillous trophoblasts, and giant cells. The cell may be a tumor or cancer cell. The cell may be an epithelial cell, an endothelial cell, or a neural cell. The cell may be non-terminally differentiated cell, for example, a stem cell (e.g., a hematopoietic stem cell, a bone marrow stem cell, a mesenchymal stem cell, a cardiac stem cell, or a neural stem cell). The cell may be living or dead. The cell may have been modified by, for example, permeabilization or cryopermeabilization, after being isolated from the subject or pharmacologically treated while alive in vitro prior to permeabilization.

The terms “living cell” or “live cell” are used herein interchangeably and refer to a cell having a biological activity in metabolism, transcription, translation, or protein synthesis.

The term “dead cell” as used herein refers to a cell without any biological activity in metabolism, transcription, translation, or protein synthesis.

The term “cell membrane component” as used herein refers to one or more constituents in a native cell membrane of a cell, with or without modification. The cell membrane component may include some or all of the constituents in a native cell membrane, for example, about 0.1-100%, 0.1-90%, 0.1-80%, 0.1-70%, 0.1-60%, 0.1-50%, 0.1-40%, 0.1-30%, 0.1-20%, 0.1-10%, 0.1-1%, 1-100%, 1-90%, 1-80%, 1-70%, 1-60%, 1-50%, 1-40%, 1-30%, 1-20%, 1-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 50-100%, 50-90%, 50-80%, 50-70%, 50-60%, 60-100%, 60-90%, 60-80%, 60-70%, 70-100%, 70-90%, 70-80%, 80-100%, 80-90% or 90-100%, 1-90%, 1-80%, 1-70%, 1-60%, 1-50%, 1-40%, 1-30%, 1-20% or 1-10% of the constituents in a native cell membrane, for example, by volume. The cell membrane component may include a receptor in the native cell membrane, and the receptor has a binding activity with a specific type of cells or cells in a specific tissue. The cell membrane component may assemble into a structure (e.g., a phospholipid bilayer) that resembles a structure in the native cell membrane. The assembly may be self-assembly.

The term “native cell membrane” as used herein refers to a naturally occurring cell membrane of a cell. The native cell membrane includes constituents such as lipids, proteins (e.g., glycoproteins), and combinations thereof.

The term “cell-derived membrane” as used herein refers to a membrane comprising a cell membrane component of a native cell membrane with modification or with an additional component. The additional component is different from the cell membrane component. The cell-derived membrane may include some or all of the of the constituents in the native cell membrane, for example, about 0.1-100%, 0.1-90%, 0.1-80%, 0.1-70%, 0.1-60%, 0.1-50%, 0.1-40%, 0.1-30%, 0.1-20%, 0.1-10%, 0.1-1%, 1-100%, 1-90%, 1-80%, 1-70%, 1-60%, 1-50%, 1-40%, 1-30%, 1-20%, 1-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 50-100%, 50-90%, 50-80%, 50-70%, 50-60%, 60-100%, 60-90%, 60-80%, 60-70%, 70-100%, 70-90%, 70-80%, 80-100%, 80-90% or 90-100%, 1-90%, 1-80%, 1-70%, 1-60%, 1-50%, 1-40%, 1-30%, 1-20% or 1-10% of the constituents in the native cell membrane. The cell-derived membrane may include a receptor having a binding activity for a specific type of cells or cells in a specific tissue. The cell-derived membrane may be formed by self-assembly of the modified cell membrane component, or a mixture of the cell membrane component, whether or not modified, and the additional component. The cell-derived membrane may comprise a structure (e.g., phospholipid bilayer) that resembles a structure in the native cell membrane. The cell-derived membrane may have a biological activity, for example, a binding activity for a specific type of cells or cells in a specific tissue, which may be, for example, about 80-120% identical to that of the native cell membrane.

The term “chimeric membrane” as used herein refers to a cell-derived membrane in which the additional component is an additional cell membrane component of an additional native cell membrane, an intracellular membrane such as a cellular membrane of an extracellular vesicle, an exosome, a secretory vesicle, a synaptic vesicle, an endoplasmic reticulum (ER), a Golgi apparatus, a mitochondrion, a vacuole or a nucleus, a bacterial membrane, a viral membrane, or a combination thereof. The cell membrane component and the additional cell membrane component may include constituents (e.g., receptors) of the same native cell membrane or different native cell membranes of cells of the same type or different types of cells in the same tissue or different tissues. The weight ratio between the cell membrane component and the additional cell membrane component may be adjusted to tune the physical and/or biological properties of the chimeric membrane, for example, a binding activity for a specific type of cells or cells in a specific tissue. The chimeric membrane may be formed by self-assembly of a mixture of the cell membrane component and the additional cell membrane component. A red blood cell membrane may be used to make a chimeric membrane.

The term “synthetic membrane” as used herein refers to a cell-derived membrane in which the additional component is a synthetic membrane component. The synthetic membrane component may be biocompatible. The synthetic membrane component may be biodegradable. The synthetic membrane component may be produced chemically, recombinantly, or both. The synthetic membrane may be formed by self-assembly of a mixture of the cell membrane component and the synthetic membrane component. The synthetic membrane may have a desirable physical and/or biological properties, for example, a binding activity with a specific type of cells or cells in a specific tissue.

The terms “targeting moiety” as used herein refers to any agent that enables a microparticle to move preferentially to one type of cells or tissues over another. The targeting moiety may be a biological molecule (e.g., peptide or protein), chemical compound or a combination thereof.

The terms “cell cytosol” and “cytoplasm” are used herein interchangeably and refer to the matrix inside of a cell.

The term “sequestering agent” as used herein refers to any molecule capable of binding a factor via a hydrogen bond, electrostatic interaction, ionically or covalently such that the factor is bound to the microparticle. The factor may be a biological molecule or structure in a subject.

The term “immunogenic” as used herein refers to any factor that when introduced into a subject causes an immune response.

The present invention provides a microparticle (MP). The MP is not naturally occurring. The MP comprises a core and a membrane surrounding the core. The membrane comprises a cell membrane component. The MP of the present invention may be capable of crossing an endothelium, which may be in a tissue (e.g., brain or lymph node). The tissue may be in a subject (e.g., human).

The MP membrane may consist of a native cell membrane of a single cell or a portion thereof. The cell membrane component may comprise some or all of the constituents in the native cell membrane, for example, about 0.1-100%, 0.1-90%, 0.1-80%, 0.1-70%, 0.1-60%, 0.1-50%, 0.1-40%, 0.1-30%, 0.1-20%, 0.1-10%, 0.1-1%, 1-100%, 1-90%, 1-80%, 1-70%, 1-60%, 1-50%, 1-40%, 1-30%, 1-20%, 1-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 50-100%, 50-90%, 50-80%, 50-70%, 50-60%, 60-100%, 60-90%, 60-80%, 60-70%, 70-100%, 70-90%, 70-80%, 80-100%, 80-90% or 90-100%, 1-90%, 1-80%, 1-70%, 1-60%, 1-50%, 1-40%, 1-30%, 1-20% or 1-10% of the constituents in the native cell membrane, for example, by volume. The native cell membrane may be obtained without modification. The cell may be a leukocyte. The leukocyte may be a lymphocyte. The lymphocyte may be a T lymphocyte. The native cell membrane may be from a leukocyte, lymphocyte or T lymphocyte.

The MP membrane may be a cell-derived membrane, a membrane from a native cell membrane. The cell-derived membrane may consist of the native cell membrane of a single cell or a portion thereof with modification. The cell-derived membrane may comprise the native cell membrane of a single cell or a portion thereof, with or without modification, and an additional component, which is not the cell component. The cell-derived membrane may include some or all of the constituents in the native cell membrane, for example, about 0.1-100%, 0.1-90%, 0.1-80%, 0.1-70%, 0.1-60%, 0.1-50%, 0.1-40%, 0.1-30%, 0.1-20%, 0.1-10%, 0.1-1%, 1-100%, 1-90%, 1-80%, 1-70%, 1-60%, 1-50%, 1-40%, 1-30%, 1-20%, 1-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 50-100%, 50-90%, 50-80%, 50-70%, 50-60%, 60-100%, 60-90%, 60-80%, 60-70%, 70-100%, 70-90%, 70-80%, 80-100%, 80-90% or 90-100%, 1-90%, 1-80%, 1-70%, 1-60%, 1-50%, 1-40%, 1-30%, 1-20% or 1-10% of the constituents in the native cell membrane, for example, by volume. The cell membrane component may comprise some or all of the constituents in the native cell membrane, for example, about 0.1-100%, 0.1-90%, 0.1-80%, 0.1-70%, 0.1-60%, 0.1-50%, 0.1-40%, 0.1-30%, 0.1-20%, 0.1-10%, 0.1-1%, 1-100%, 1-90%, 1-80%, 1-70%, 1-60%, 1-50%, 1-40%, 1-30%, 1-20%, 1-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 50-100%, 50-90%, 50-80%, 50-70%, 50-60%, 60-100%, 60-90%, 60-80%, 60-70%, 70-100%, 70-90%, 70-80%, 80-100%, 80-90% or 90-100%, 1-90%, 1-80%, 1-70%, 1-60%, 1-50%, 1-40%, 1-30%, 1-20% or 1-10% of the constituents in the native cell membrane, for example, by volume. The cell-derived membrane may include a receptor having a binding activity for a specific type of cells or cells in a specific tissue. The cell-derived membrane may be a chimeric membrane where the additional component is an additional cell membrane component or a synthetic membrane where the additional component is a synthetic membrane component. The synthetic membrane component may comprise phosphatidylcholine, phosphatidylethanolamine, phosphatidylserine, phosphatidylinositol, phosphatidylglycerol, pphingomyelin, dimyristoyl phosphatidylglycerol sodium salts, phosphatidic acid, lyosphospholipids, oxidized phospholipids, sterols, proteins, glycoproteins, receptors and transporters.

In one embodiment, the MP membrane is from a permeabilized cell. The permeabilized cell may be a permeabilized leukocyte, lymphocyte or T lymphocyte. The cell-derived membrane may comprise the cell membrane of the permeabilized cell or a portion thereof. The permeabilized cell may have been subject to cryopermeabilization, a detergent, or a chemical permeabilization solution.

The permeabilized cell membrane may comprise some or all of the constituents of the native cell membrane of the corresponding cell used to prepare the permeabilized cell, for example, about 0.1-100%, 0.1-90%, 0.1-80%, 0.1-70%, 0.1-60%, 0.1-50%, 0.1-40%, 0.1-30%, 0.1-20%, 0.1-10%, 0.1-1%, 1-100%, 1-90%, 1-80%, 1-70%, 1-60%, 1-50%, 1-40%, 1-30%, 1-20%, 1-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 50-100%, 50-90%, 50-80%, 50-70%, 50-60%, 60-100%, 60-90%, 60-80%, 60-70%, 70-100%, 70-90%, 70-80%, 80-100%, 80-90% or 90-100%, 1-90%, 1-80%, 1-70%, 1-60%, 1-50%, 1-40%, 1-30%, 1-20% or 1-10% of the constituents of the native cell membrane of the corresponding cell, for example, by volume. The cell membrane component in the cell-derived membrane comprise about 0.1-100%, 0.1-90%, 0.1-80%, 0.1-70%, 0.1-60%, 0.1-50%, 0.1-40%, 0.1-30%, 0.1-20%, 0.1-10%, 0.1-1%, 1-100%, 1-90%, 1-80%, 1-70%, 1-60%, 1-50%, 1-40%, 1-30%, 1-20%, 1-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 50-100%, 50-90%, 50-80%, 50-70%, 50-60%, 60-100%, 60-90%, 60-80%, 60-70%, 70-100%, 70-90%, 70-80%, 80-100%, 80-90% or 90-100%, 1-90%, 1-80%, 1-70%, 1-60%, 1-50%, 1-40%, 1-30%, 1-20% or 1-10% of the constituents of the native cell membrane of the corresponding cell, for example, by volume.

The MP membrane may be self-assembled by a cell membrane component, optionally with an additional component. The MP membrane may be prepared by mixing the cell membrane component and the additional component. The composition of the MP membrane may be adjusted to tune the physical and/or biological properties of the MP. The MP membrane may comprise a structure (e.g., phospholipid bilayer) that resembles a structure in a native cell membrane. The MP membrane may have a biological activity, for example, a binding activity for a specific type of cells or cells in a specific tissue similar or identical to that of a native cell membrane.

In the MP, the cell membrane component may be present at about 0.1-100%, 0.1-90%, 0.1-80%, 0.1-70%, 0.1-60%, 0.1-50%, 0.1-40%, 0.1-30%, 0.1-20%, 0.1-10%, 0.1-1%, 1-100%, 1-90%, 1-80%, 1-70%, 1-60%, 1-50%, 1-40%, 1-30%, 1-20%, 1-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 50-100%, 50-90%, 50-80%, 50-70%, 50-60%, 60-100%, 60-90%, 60-80%, 60-70%, 70-100%, 70-90%, 70-80%, 80-100%, 80-90% or 90-100%, 1-90%, 1-80%, 1-70%, 1-60%, 1-50%, 1-40%, 1-30%, 1-20% or 1-10%, based on the total amount, for example, volume, of the membrane.

In the MP, the membrane may further comprise a targeting moiety. The targeting moiety may comprise an integrin, selectin, cadherin, immunoglobulin-like adhesion molecule, addressin, chemokine receptor, chemokine ligand, growth factor receptor, immunoglobulin superfamily protein (e.g., toll-like receptor (TLRS), T cell receptor (TCR), B cell receptor (BCR), major histocompatibility complex (MHC) molecule), ion channel-linked receptor, G protein-coupled receptor, enzyme-linked receptor, antibody or a fragment thereof (e.g., nanobody), or a binding domain of any of these moieties. The targeting moiety may be on the outer surface of the membrane. The targeting moiety may be a constituent of a native cell membrane. The targeting moiety may have a specific binding affinity with a specific type of cells or cells in a specific type of tissues. The targeting moiety may have a specific binding affinity with endothelium in brain, and examples of such target moieties include CCR7, CXCR3, L-selectin, P-selectin glycoprotein ligand 1 (PSGL1), VLA-4, LFA-1, CCR6. The targeting moiety may have a specific binding affinity with endothelium in a lymph node, and examples of such target moieties include L-selectin, Lymphocyte function-associated antigen 1 (LFA-1), chemokine (C-C motif) receptor 7 (CCR7), Integrin a401 (VLA-4), lysophosphatidic acid receptors (LPA2, LPA5, LPA6) Where the MP membrane consists of a native cell membrane of a single cell or a portion thereof, the targeting moiety may be a constituent of the native cell membrane. The cell membrane component may comprise the targeting moiety.

Where the MP membrane is a cell-derived membrane from a native cell membrane, the targeting moiety may be a constituent of the native cell membrane. The cell membrane component may comprise the targeting moiety.

In the MP, the core may be in the form of a liquid, a solid or a combination thereof. The core may be biocompatible. The core may be biodegradable. The core may comprise cytoplasm, which may be native or modified. The cytoplasm may be of the same cell or a cell of the same type as the cell of which the native cell membrane is in the MP membrane or from which the cell-derived membrane is in the MP membrane.

The liquid core may comprise an aqueous solution, an oil or a combination thereof. The liquid may be doped with viscosity-modifying agents such as dextran and hyaluronic acid to tune liquid viscosity and regulate payload loading and release from the MP. A liquid core may contain multiple aqueous solutions, multiple oil solutions, or both aqueous and oil. Multiple liquid phases within an MP may be structured, for example, core-(multi-)shell arrangements wherein alternating layers of immiscible phases are oriented and/or exist as double emulsions with many discrete phases existing of one immiscible fluid within the other.

The core may comprise a polymer, which may be natural or synthetic. The core may comprise an extracellular matrix protein, which may be purified, recombinant, or decellularized. The polymeric core may comprise a synthetic polymer such as PEG, PLGA, and a combination thereof, a natural polymer such as alginate and collagen, and/or soluble extracellular matrix (ECM) proteins isolated from a tissue or cell line (e.g., matrigel). The ECM proteins may be a secreted, purified or recombinant proteins found in or derived from ECM proteins found in a tissue from various mammalian species, for example, human, non-human primates, porcine, equine, lampine, and rodents.

In the MP, the core may have a size in the range of about 0.1-1,000 μm, 0.1-900 μm, 0.1-800 μm, 0.1-700 μm, 0.1-600 μm, 0.1-500 μm, 0.1-400 μm, 0.1-300 μm, 0.1-200 μm, 0.1-100 μm, 0.1-50 μm, 0.1-10 μm, 0.1-1 pm, 0.5-1,000 μm, 0.5-900 μm, 0.5-800 μm, 0.5-700 μm, 0.5-600 μm, 0.5-500 μm, 0.5-400 μm, 0.5-300 μm, 0.5-200 μm, 0.5-100 μm, 0.6-1,000 μm, 0.6-900 μm, 0.6-800 μm, 0.6-700 μm, 0.6-600 μm, 0.6-500 μm, 0.6-400 μm, 0.6-300 μm, 0.6-200 μm, 0.6-100 μm, 0.7-1,000 μm, 0.7-900 μm, 0.7-800 μm, 0.7-700 μm, 0.7-600 μm, 0.7-500 μm, 0.7-400 μm, 0.7-300 μm, 0.7-200 μm, 0.7-100 μm, 0.8-1,000 μm, 0.8-900 μm, 0.8-800 μm, 0.8-700 μm, 0.8-600 μm, 0.8-500 μm, 0.8-400 μm, 0.8-300 μm, 0.8-200 μm, 0.8-100 μm, 0.9-1,000 μm, 0.9-900 μm, 0.9-800 μm, 0.9-700 μm, 0.9-600 μm, 0.9-500 μm, 0.9-400 μm, 0.9-300 pm, 0.9-200 μm, 0.9-100 μm, 1-1,000 μm, 1-900 μm, 1-800 μm, 1-700 μm, 1-600 μm, 1-500 μm, 1-400 μm, 1-300 um, 1-200 μm, 1-100 μm, 100-1,000 μm, 100-900 μm, 100-800 μm, 100-700 μm, 100-600 μm, 100-500 μm, 100-400 μm, 100-300 μm, 100-200 μm, 500-1,000 μm, 500-900 μm, 500-800 μm, 500-700 μm, 500-600 μm, 750-1,000 μm, 750-900 μm or 750-800 μm. For example, the MP may have a size of 0.8-500 μm.

The core may comprise an active agent, which is also known as a payload. The active agent may comprise a biological molecule, a chemical compound, or a combination thereof. The active agent may comprise a nanoparticle (e.g., metallic particle, polymeric particle, dendrimer particle, or inorganic particle), a liposome, a virus, or a combination thereof. The active agent may have a biological activity, for example, a therapeutic effect. The active agent may comprise a therapeutic, an imaging agent, a sequestering agent, a prophylactic agent, a diagnostic agent, a prognostic agent, an excipient or a combination thereof.

The core may be prepared from a cell. Such a core may comprise some or all of the cytoplasm of the cell, for example, about 0.1-100%, 0.1-90%, 0.1-80%, 0.1-70%, 0.1-60%, 0.1-50%, 0.1-40%, 0.1-30%, 0.1-20%, 0.1-10%, 0.1-1%, 1-100%, 1-90%, 1-80%, 1-70%, 1-60%, 1-50%, 1-40%, 1-30%, 1-20%, 1-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 50-100%, 50-90%, 50-80%, 50-70%, 50-60%, 60-100%, 60-90%, 60-80%, 60-70%, 70-100%, 70-90%, 70-80%, 80-100%, 80-90% or 90-100%, 1-90%, 1-80%, 1-70%, 1-60%, 1-50%, 1-40%, 1-30%, 1-20% or 1-10% of the cytoplasm of the cell, for example, by volume. The cell may be a leukocyte (e.g., lymphocyte).

The MP is biocompatible, and may be biodegradable. The MP may not be immunogenic.

For each MP of the present invention, a method for transporting the MP is provided. The transport method comprises administering the MP to an endothelium, whereby the microparticle is bound to the endothelium. The transport method further comprises moving the microparticle across the endothelium.

According the transport method, the MP comprises a core and a membrane surrounding the core, and the membrane comprises a cell membrane component. The membrane may further comprise an additional component. The membrane may consist of a native cell membrane. The membrane may comprise a cell-derived membrane. The cell-derived membrane may be a chimeric membrane or a synthetic membrane. The membrane may comprise a targeting moiety. The targeting moiety may comprise an integrin, selectin, cadherin, immunoglobulin-like adhesion molecule, addressin, chemokine receptor, chemokine ligand, growth factor receptor, immunoglobulin superfamily protein (e.g., toll-like receptor (TLRS), T cell receptor (TCR), B cell receptor (BCR), major histocompatibility complex (MHC) molecule), ion channel-linked receptor, G protein-coupled receptor, enzyme-linked receptor, antibody or a fragment thereof (e.g., nanobody), or a binding domain of any of these moieties. The endothelium may be in a subject. The subject may be a human or non-human. The MP may be administered intravenously to the subject.

The transport method may further comprise moving the MP to a target site after moving the MP across the endothelium. The target site is site in a tissue or organ, to which the MP goes. The endothelium in such a tissue or organ may have a unique receptor profile that interacts with the MP and moves the MP across the endothelium. The unique endothelial profile may change by region in the body of the subject due to a disease. The target site may be in the tissue on the other side of the endothelium. The target site may be in lymph node (LN) lobule, brain parenchyma, tissue interstitium or tissue parenchyma. The target site may be in a lymph node (LN), central nervous system (CNS), gut-associated lymphoid tissue, teste, lung, tumor site (e.g., tumor-associated macrophages (TAMS) or tumor-associated Lymphocytes (TALs)), or site of inflammation.

According to the transport method, the MP may be circulated from a blood stream or lymph fluid across an endothelium into the surrounding interstitium or tissue. The endothelium may be in brain or a lymph node. For example, the endothelium may be in a lymph node and the target site may be a lobule in the lymph node. The endothelium may be in a brain and the target site may be in brain parenchyma or cerebrospinal fluid (CSF). Both the endothelium and the target site may be in a tumor. The core may comprise an active agent, and the transport method may further comprise releasing the active agent at the target site. The active agent may comprise a biological molecule, a chemical compound, or a combination thereof. The active agent may comprise a nanoparticle (e.g., metallic particle, polymeric particle, dendrimer particle, or inorganic particle), a liposome, a virus, or a combination thereof. The active agent may have a biological activity, for example, a therapeutic effect. The active agent may comprise a therapeutic, an imaging agent, a sequestering agent, a prophylactic agent, a diagnostic agent, a prognostic agent, an excipient or a combination thereof.

The transport method may further comprise sequestering a molecule by the MP from the target site. The MP may comprise a sequestering agent. The sequestering agent may be in the core, the membrane or both.

The transport method may further comprise causing a biological response at the target site. The biological response may be initiated at the endothelium, and include moving the MP across the endothelium. The biological response may be initiated in the tissue or interstitium or parenchyma on the other side of the endothelium. The biological response may selected from the group consisting of immune interactions, cancer therapy, vaccine responses, and immunotherapy.

For each MP of the present invention, a method for preparing the MP is provided. The preparation method comprises mixing a core with a membrane. The membrane comprises a cell membrane component. The prepared MP comprises a core and a membrane surrounding the core, and the membrane comprises a cell membrane component. The preparation method may further comprise mixing the cell membrane component with the core. The cell membrane component may be present at about 0.1-100%, 0.1-90%, 0.1-80%, 0.1-70%, 0.1-60%, 0.1-50%, 0.1-40%, 0.1-30%, 0.1-20%, 0.1-10%, 0.1-1%, 1-100%, 1-90%, 1-80%, 1-70%, 1-60%, 1-50%, 1-40%, 1-30%, 1-20%, 1-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 50-100%, 50-90%, 50-80%, 50-70%, 50-60%, 60-100%, 60-90%, 60-80%, 60-70%, 70-100%, 70-90%, 70-80%, 80-100%, 80-90% or 90-100%, 1-90%, 1-80%, 1-70%, 1-60%, 1-50%, 1-40%, 1-30%, 1-20% or 1-10%, based on the total amount, for example, volume, of the membrane.

The membrane may further comprise an additional component, and the preparation may further comprise mixing the cell membrane component, the core and the additional component. The membrane may be a chimeric membrane, in which the additional component is an additional cell membrane component. The membrane may be a synthetic membrane, in which the additional component is a synthetic membrane component.

In one embodiment, the membrane is a cell membrane of a permeabilized cell, and the preparation method comprises adding a core into a permeabilized cell. The permeabilized cell may be a permeabilized leukocyte, lymphocyte or T lymphocyte. The permeabilized cell may have been subject to cryopermeabilization, a detergent, or a chemical permeabilization solution.

In another embodiment, the membrane is a cell membrane of a permeabilized leukocyte, and the preparation method further comprises injecting or using concentration gradients to diffuse a material into the permeabilized leukocyte. The material may further be manipulated with a chemical or photoactivation method to undergo a sol-gel transition.

In yet another embodiment, the preparation method further comprises wrapping the core with the membrane. The membrane may be a cell membrane of a permeabilized cell. The permeabilized cell may be a permeabilized leukocyte, lymphocyte or T lymphocyte. The permeabilized cell may have been subject to cryopermeabilization, a detergent, or a chemical permeabilization solution.

The preparation method may further comprise loading the core with an active agent. The active agent may comprise a biological molecule, a chemical compound, or a combination thereof. The active agent may comprise a nanoparticle (e.g., metallic particle, polymeric particle, dendrimer particle, or inorganic particle), a liposome, a virus, or a combination thereof. The active agent may have a biological activity, for example, a therapeutic effect. The active agent may comprise a therapeutic, an imaging agent, a sequestering agent, a prophylactic agent, a diagnostic agent, a prognostic agent, an excipient or a combination thereof.

The preparation method may further comprise preparing the core from a permeabilized cell. The permeabilized cell may be a permeabilized leukocyte, lymphocyte or T lymphocyte. The permeabilized cell may have been subject to cryopermeabilization, a detergent, or a chemical permeabilization solution.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### EXAMPLE 1

### T-Cell Transport Through High Endothelial Veins in the Lymph Node

To determine the mechanism of cell trafficking across endothelial barriers, murine lymph nodes were collected, sectioned to 200 μm via vibratome, and fixed using 2% Paraformaldehyde, 2% Glutaraldehyde with 2 mM CaCl₂ in 0.1M Na Cacodylate Buffer. Following a multi-step process for adding 2% osmium tetroxide to the samples for contrast, samples are embedded in a Durcupan resin block and imaged using a serial-block face scanning electron microscope (SBF-SEM). During SBF-SEM imaging, samples were mounted and serially sectioned and imaged at Z step of 10 nm. This collected image stack was analyzed for T cell crossing into the lobule interactions, showing clear engulfment of T cells by the endothelium (FIG. 2).

### EXAMPLE 2

### Creating Permeabilized Dead Cells as MPs for Drug Release

MPs (termed CSTL herein) may be produced from dead T lymphocytes (or any other cell line). These CSTLs may be loaded via diffusion with any dye for visualization, filled with a hydrogel, or loaded with any other payload (e.g., drug). The CSTLs may be intravenously delivered in mice or human individuals for targeted uptake in a number of organ, and cross endothelial barriers.

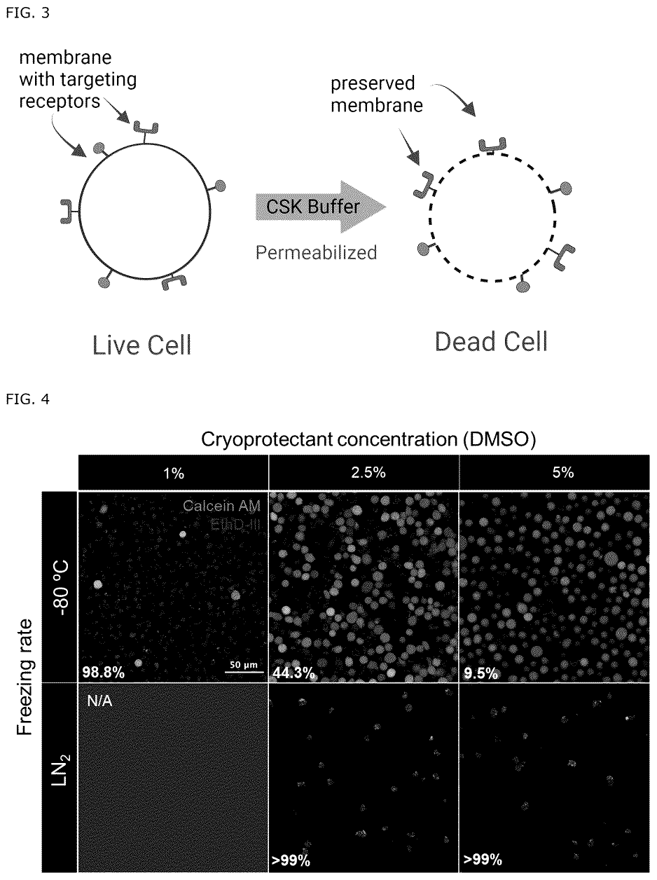

As illustrated in FIG. 3, freshly isolated cultured cells (live cells) having a cell membrane with targeting receptors may be treated with a cytoskeletal stabilizing buffer (CSK) to produce permeabilized dead cells having a preserved membrane with the targeting receptors. For example, after isolation, the samples were rinsed with 25 mL of warm phosphate-buffered saline supplemented with 0.01% Tween 20 (PBS-T) and immediately immersed into a modified, ice-cold cytoskeleton stabilizing buffer (CSK) (10 mM HEPES, 0.5% Triton X-100, 300 mM sucrose, 3 mM MgCl₂, and 50 mM NaCl in DI H₂O) for 1 min. The samples were removed from the CSK and immediately submersed into ice-cold 4% paraformaldehyde in PBS-T and placed in a 37° C. water bath for 10 min. The samples were rinsed with 25 mL of warm PBS-T followed. For cryopermeabilization, the freshly isolated cultured cells were resuspended in vials having PBS with 5% DMSO at a cell density of 1×10⁷mL⁻¹, and the cell vials were submerged in liquid nitrogen (LN₂) for 12 hours prior to use. Alternatively, the freshly isolated cultured cells were resuspended in vials having PBS with 1% DMSO and the cell vials were frozen at −80 degrees Centigrade for 12-18 hours prior to use.

To test the impacts of permeabilizing on primary cells, a range of freezing temperatures (−20, −80, LN₂) and cryoprotectant concentrations (DMSO 0.1-10%) were used and assessed for cell death via using Live/Dead viability staining and Trypan blue permeability test (FIG. 4). Freezing in LN₂ in 5% DMSO for 12 hours offered >99% cell death with the most optimal stability. In principle, any method that permeabilizes the cell membrane and causes death while maintaining the overall structure of the cell as defined by maintaining the approximate diameter, circularity, and morphology would be compatible with these methods. Additionally, hydrogel prepolymers such as alginate can diffuse into permeabilized cells and after a solvent exchange, can undergo a sol-gel transition with the addition of CaCl₂ to the bath to form an inner hydrogel core.

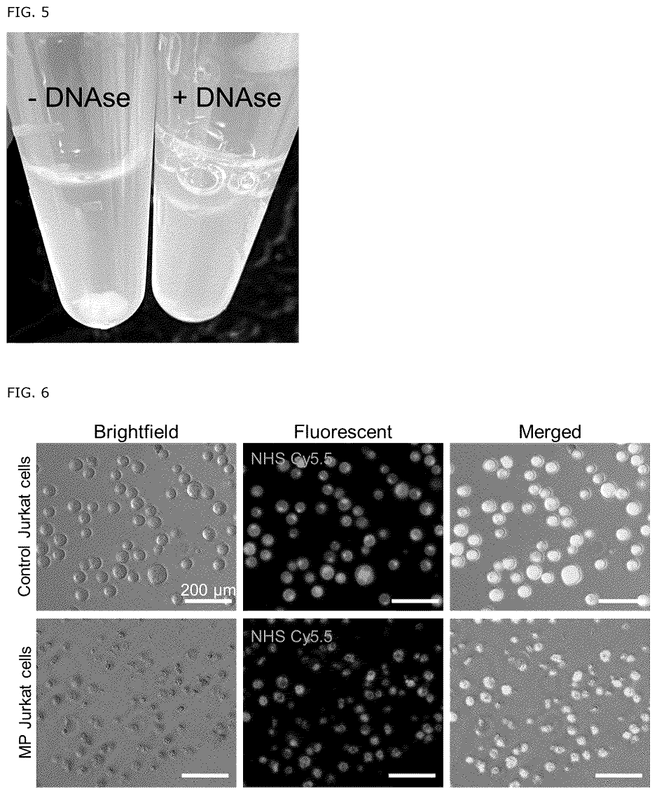

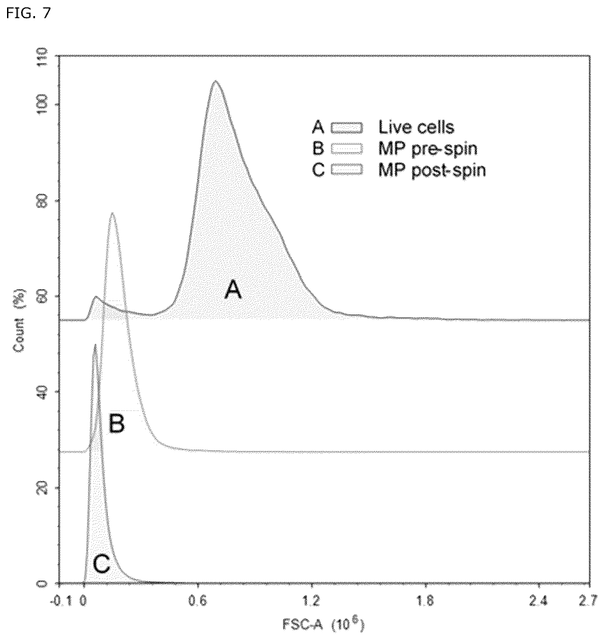

Stability and process testing: DNAse concentrations (0.1-25 μg/ml), temperature (4-37° C.), and repeated centrifuge spins (2-3×) were assessed. Impacts to the vehicle were measured using fluorescence microscopy, hemacytometer, and flow cytometry. DNAse addition was essential for successful MP resuspension (FIG. 5). Following treatment, cells maintain their individual morphology and structure; however, the cells have altered optical and fluorescent properties relative to live cells (FIG. 6). Lastly, flow cytometry data (FIG. 7) combined with imaging details reveal that the live cells are altered and/or modified through the permeabilization process, yet remain intact as individual MPs. These cells can be used in this state or filled with a core (e.g., oil, viscous aqueous solutions, and/or hydrogel solutions that are subsequently induced to undergo a sol-gel transition via thermal, chemical or photochemical methods.

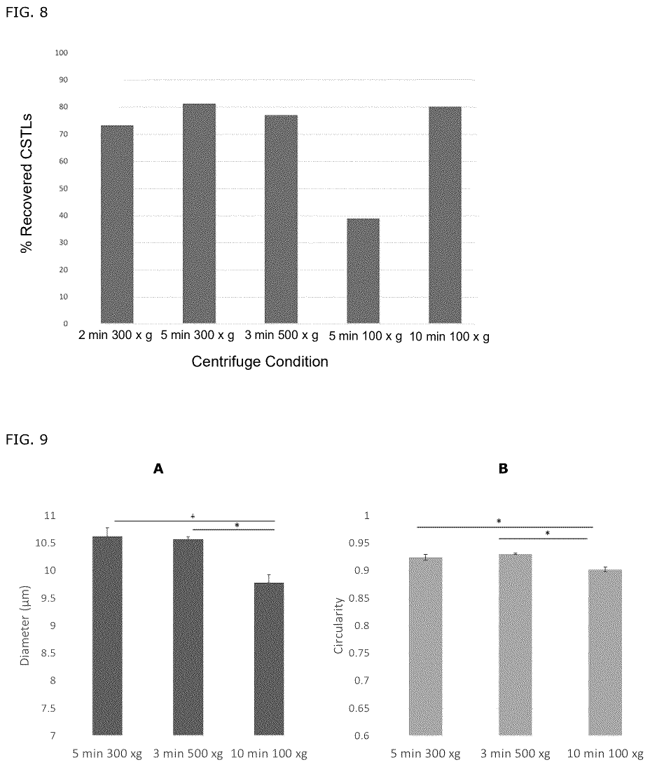

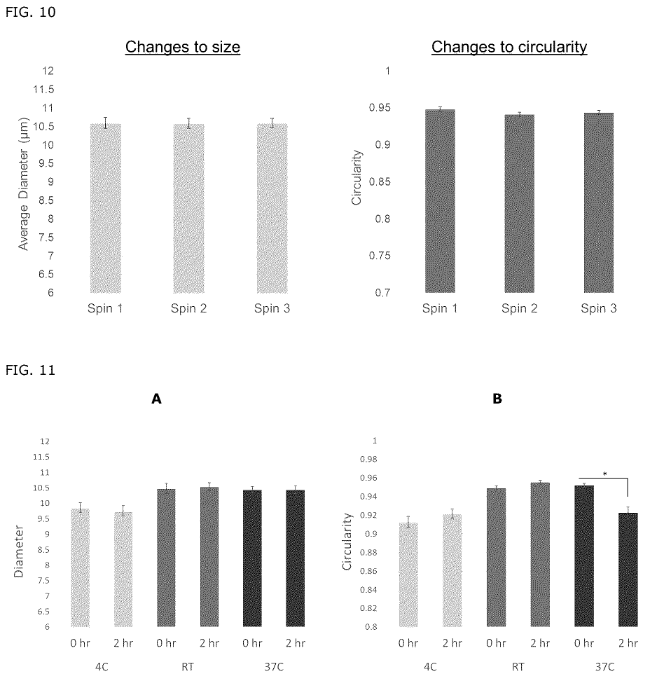

Assessment of stability from centrifugation and under flow: To test the handling and recovery of these MPs on MP stability, a range of centrifuge speeds (e.g., 100-1000×g for 10 s-10 min) were tested. As shown in the centrifugation experiments, centrifuge speeds between 300-500×g for 2-3 minutes allowed for maximal retention of MPs (FIG. 8) and minimal changes in diameter and circularity (FIG. 9). No changes to size or circularity were observed for repeated (up to 3 sequential spins) at 500×g for 3 minutes (FIG. 10). Following this, the diameter and circularity of MPs stored at different temperatures for 2 hrs were measured to determine refrigeration or room temperature storage conditions minimize variability/stability of MPs (FIG. 11).

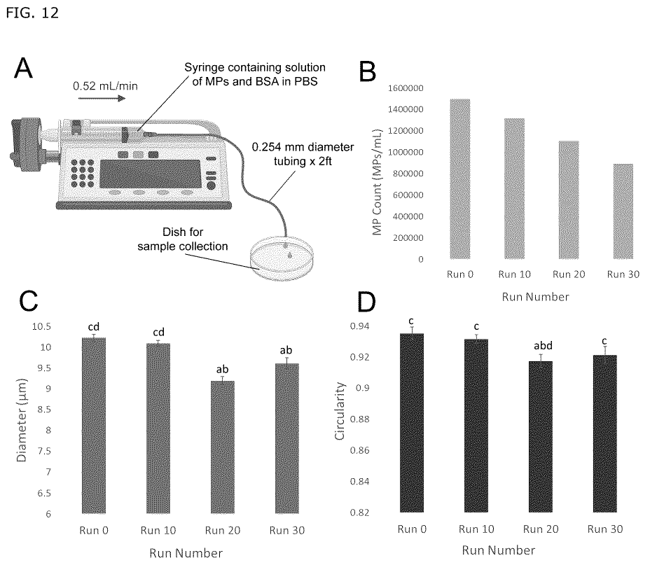

Finally, impacts of fluid flow on vehicle breakdown were tested at 37° C. via rocker or microfluidic blood vessel mimic at 0.52 mL/min (FIG. 12). Following testing of MPs under flow, about a 40% reduction in MP count was observed after 30 runs through a 0.61 meter blood vessel mimic (FIG. 8).

MP storage: experiments were performed by creating MPs from permeabilized cells and stored them for varying lengths of time. MPs may be stored in LN₂ for up to 6 weeks and thawed for use without significant degradation of MP structure and function. This extended storage potential of permeabilized MPs allows for translational capabilities.

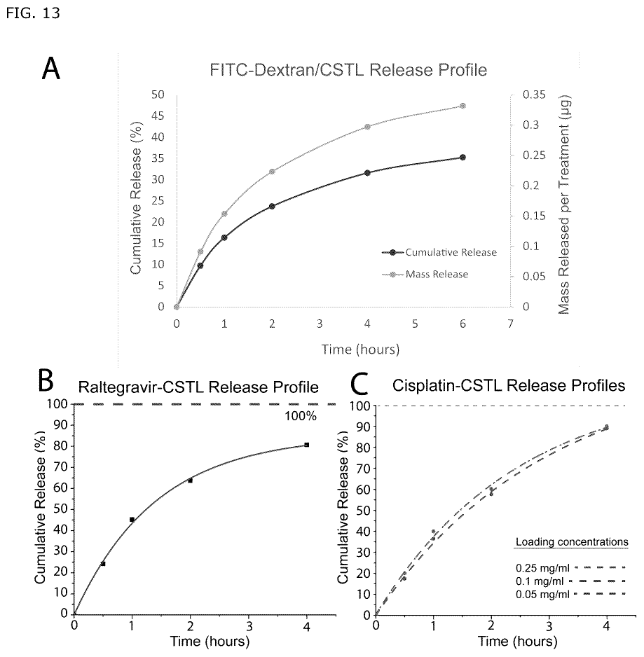

MP drug loading: MPs were incubated with a range of cisplatin concentrations (0.05-0.5 mg/ml) at 37° C. for 1 hour for passive uptake of the drug. Following incubation, half of the sample was centrifuged and the pellet resuspended in RIPA buffer. This pellet fraction was analyzed via mass spectroscopy (MS) to determine the theoretical maximum loading of drug per MP. The remainder of the sample was centrifuged and resuspended in fresh PBS. The sample was added to the top of a transwell insert for a longitudinal release kinetics study, with ICP-MS performed on the sampled basal solution collected at t=0, 0.5, 1, 2, 4, 8, 12, and 24 hours. Similar studies were conducted with Raltegravir and FITC-dextran with MS or fluorescence measurements. As shown in these studies, permeabilized cell MPs successfully released a number of payloads with predictable release kinetics similar to synthetic drug carrier systems. For small molecules, such as Cisplatin and Raltegravir, roughly 80-90% of the drug is released after 4 hours (FIG. 13). For a larger molecule, such as 70 kDa FITC-Dextran, release kinetics are slowed to roughly 35% of total cargo being released at 8 hours.

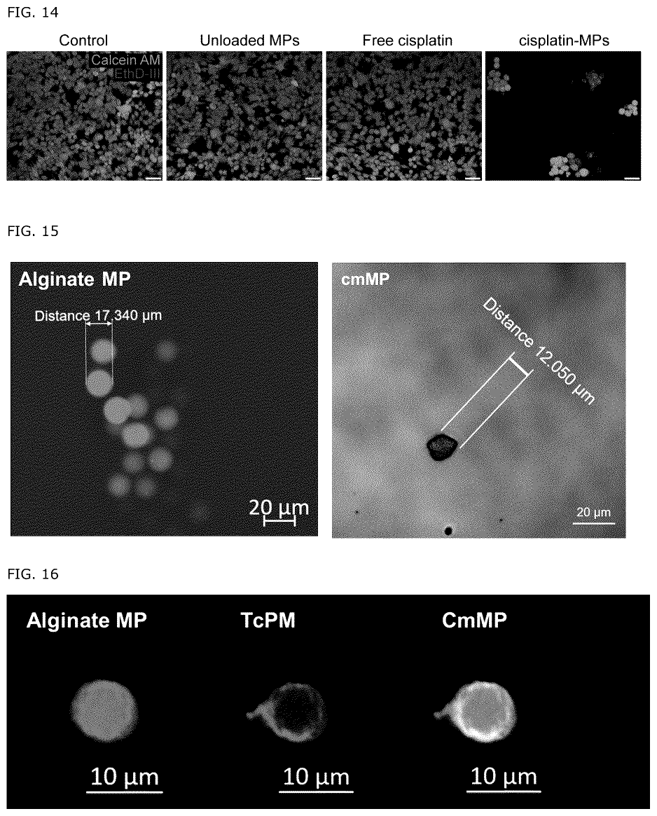

In vitro validation of released drug efficacy: Using release profiles as discussed previously, 4T1-luc2 cells were treated with cisplatin loaded MPs and compared to free drug treatment. To calculate the corresponding free drug dose, cumulative release of cisplatin was determined at 4 hours, and added to the cells. Additional controls include unloaded MPs and untreated cells. Free cisplatin and unloaded MPs led to little to no cell death as compared to control after 4 hours (FIG. 14). Cisplatin loaded MPs led to significant cell death as seen by changes to morphology and regions of clear cell detachment. As shown in these experiments, the local release of high levels of payload may result in a stark increase in efficiency for the treatment of diseased cells.

### EXAMPLE 3

### Making Membrane-Wrapped Sodium Alginate MPs Derived from T Lymphocytes

General approach to generate membrane-wrapped alginate microparticles (MPs): The preparation method to synthesize biomembrane-covered microparticles (MPs), also known as cell-mimetic microparticles (cmMPs), consist of a hydrogel “core” that is independently produced and subsequently wrapped with membranes isolated from T lymphocytes. Micron-sized sodium alginate hydrogels were made with a microfluidic droplet generator using a flow-focusing configuration to achieve high monodispersity with fine control of hydrogel size. Sodium alginate crosslinks in the presence of divalent cations to undergo sol-gel transition. Monodisperse liquid alginate droplets were produced at a rate of thousands to tens of thousands per second in a single microfluidic device by tuning the flow rates of the continuous phase (oil) to the disperse phase (sodium alginate and Ca-EDTA). With the addition of 0.2% acetic acid to the oil phase to enable a sol-gel transition. The subsequent alginate hydrogel microsphere “cores” underwent a solvent exchange to remove the oil phase (FIG. 15). Separately, T lymphocyte membrane extraction was accomplished using standard procedures via an osmosis-based mild hypotonic cell lysis solution coupled with physical homogenization followed by differential centrifugation and ultracentrifugation steps to obtain concentrated isolated plasma membranes. The purified membranes and alginate cores were mixed and co-extruded through a polycarbonate membrane of pore size ranging from 8 μm to 20 μm depending upon the desired MP size. The wrapped MPs were subsequently characterized for size (monodispersity), zeta potential, and specific membrane protein compositions using DLS, zetasizer, and flow cytometry (FIG. 16).

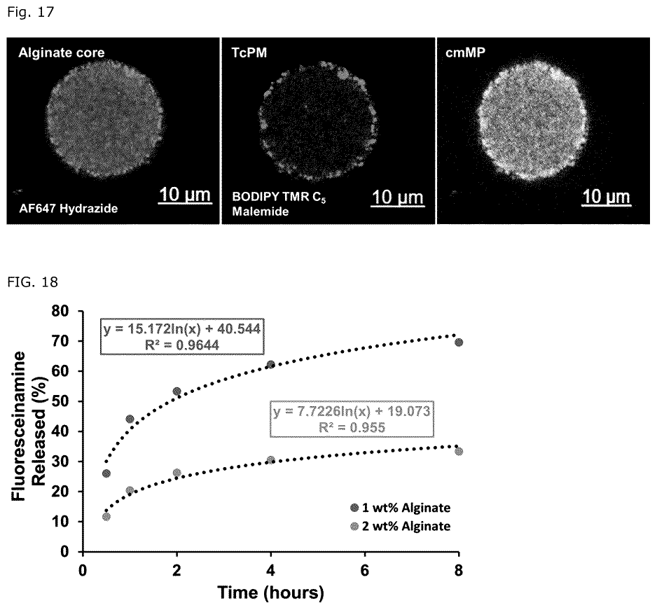

Importantly, this platform can be easily adapted with straightforward chemistry. For visualization of the MP during in vitro and in vivo experiments, sodium alginate has been covalently conjugated with different fluorescent labels such as FITC, TRITC, and DAPI via carbodiimide crosslinking. Similarly, MP membrane visualization will be achieved through covalent coupling to bioreactive dyes such as Cy5.5 NHS ester and BODIPY TMR C₅ maleimide⁸⁵ or membrane dyes such as DiD and DiO (FIGS. 16 and 17). For drug payloads that consist of large molecules, like antibodies, the therapeutic was mixed with the sodium alginate solution before forming the hydrogel cores. For small diffusible molecules, MPs were soaked in a drug solution until fully saturated prior to administration or testing.

Tuning release kinetics and MP degradation to enable sustained-release formulations: small molecule fluorophores, HIV antiretrovirals (ARVs) including tenofovir disoproxil fumarate and darunavir, gold nanoparticles, and antibodies were used to quantify release kinetics of two distinct classes of exemplar therapeutic compounds (payload) in vitro. For small molecules (fluorophores and ARVs), MPs were soaked until saturated in a solution of the payload. For large molecules (antibodies and 80 nm gold nanoparticles (NPs)), the payload was added to the alginate solution prior to forming the MP core. MP alginate core weight percent was modulated to quantify drug release kinetics. MPs were placed into the top reservoir of a transwell in water and the basal compartment was sampled over time in an incubator rocker. As expected, release kinetics from membrane-wrapped MPs follow expected release kinetics with higher mass fractions increasing the release half-life (FIG. 18).

The MP membrane and formulation may be independently modulated to engineer payload loading and release. Sterol concentration is known to affect liposome and membrane stability and drug release, with increasing concentration increasing stability and slowing release kinetics. Purified membranes may be supplemented with sterols. Similarly, MP core formulations may be varied by using numerous standard approaches to tune the resultant pore size, for example, varying the mass fraction of the sodium alginate and altering the extent of cross-linking within the resulting hydrogel core. Additionally, spatial gradients of pore sizes along the radius may be induced to create a core-shell organization within a single MP “core” by generating MP cores as described above and performing a secondary cross-linking procedure in a CaCl₂ bath. This secondary diffusive cross-linking wave from the core surface into the core enables the sculpting of the release kinetics into multiple phases, in particular, slowing the initial release of the payload. Serial cross-linking procedures may be used to generate multiple “shells” of decreased porosity within the MP core. These refinements may be used to slow the first-phase and second-phase release kinetics, parameters with the highest impact on decreasing the off-target loss of drug payload and extending the release of the drug in the target tissues compared to the current formulation's performance according to PK model analysis.

Conclusions: The physicochemical properties of the alginate core, such as the degree of crosslinking and weight fraction of alginate, govern alginate pore size and degradation and, in turn influence key properties of the formulation such as cargo loading efficiency and release kinetics. For small-molecule drugs, the alginate cores act as a sponge, and the payload elutes through diffusion-like processes; our data shows that this mechanism enables payload elution half-lives on the order of weeks. For larger molecules such as antibodies, the payload may be trapped within the core hydrogel matrix, and extended-release may occur over a month or longer as the core disintegrates. Smaller molecules chemically conjugated to the core may also have these extended-release kinetics. Our data show that both the MP disassociation rate and the elution rate of its integrated payload may be tunable material properties of the core. The particles may therefore be optimized using several approaches detailed below to achieve sustained release of the drug into target tissues for an extended period of time.

### EXAMPLE 4

### MP Transport and Tissue Localization in a Mouse Lymph Node

MP Injection and LN Homing in a mouse model: T lymphocytes were isolated from the spleen of a CD1 mouse via a commercial kit. The cells were permeabilized by freezing. To label the MPs for visualization in tissue, the MPs were thawed at 4° C. and incubated for 20 minutes with 78 μM NHS Cy5.5 in 1×PBS. This dye labels amino-groups remaining in the cell post cryopermeabilization. CD-1 mice were injected with a dose of 1.5×10⁶ cells intravenously and euthanized at distinct time points (t=0, 1, 2, 4, 8, 16, and 24 hours). Blood samples were collected at each time point via intracardiac puncture, to quantify CSTLs remaining in circulation. Brachial, inguinal, and popliteal LNs were isolated, fixed overnight in 4% paraformaldehyde (PFA), and embedded in 6% agarose gel for vibratome sectioning. The 200 μm sections will then be imaged to assess CSTL uptake.

Validation that membrane composition regulates specific processes for MP accumulation in tissue by crossing the endothelial barriers: To validate targeting and uptake of MPs is specific, L-selectin antibody blocking was used to confirm functional LN extravasation. L-selectin is a glycoprotein expressed on T lymphocyte membranes and is responsible for initial tethering and rolling on high endothelial venules (HEVs). Thus, blocking this interaction is expected to prevent extravasation into the lobule. In the experimental group, mice were injected with 0.4 mg/kg of L-selectin antibody and with a dose of MPs. In the control group, mice only received the MPs. At 2 hours post cell injection, mice were euthanized and vibratome sections will be prepared as previously described. CSTL uptake in both sections will be compared by enumerating lobule entry.

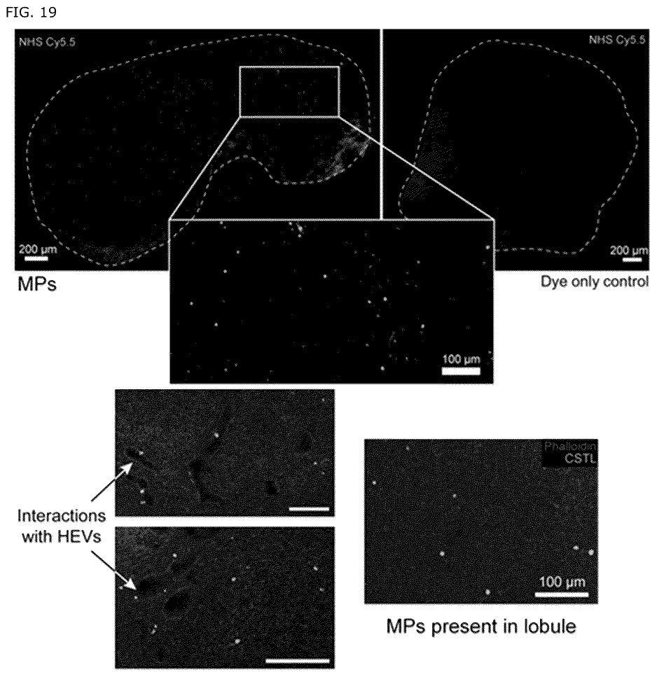

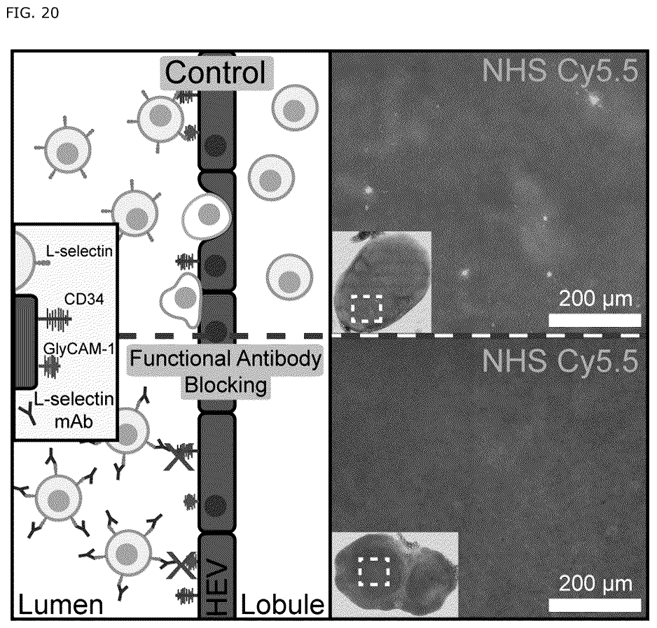

Conclusions for LN targeting: From the experiments, it is clear that the delivery of permeabilized T cell MPs was a viable approach for targeting and entering the lymph node lobule. MPs were able to survive blood flow and bind to the HEVs within the lymph node (FIG. 19). While T lymphocyte transendothelial migration has been thought of as an active process and evidence supports the motile capacity of lymphocytes, other leukocyte-endothelial cell interactions may also act as possible modes of crossing a biological barrier. Additionally, a recent study has identified “HEV pockets”, a temporary holding site in the HEV lumen for transient lymphocyte migration restraint. This interaction may control the rate of lymphocyte ingress to balance the egress via lymphatics. Further this process is specific to the membrane composition as L-selectin functional antibody blocking prevented MP extravasation into the tissue (FIG. 20) similarly to live T cells. It is clear that the HEV endothelial cells play a major role in the passage of lymphocytes across the barrier, and therefore their role in MP entry into the lymph node may be paramount. Overall, permeabilized T cell MPs have the capacity to be loaded with and release drug and enter the lymph node lobule, offering a powerful tool for LN targeted drug delivery.

### EXAMPLE 5

### MP Transport and Tissue Localization in a Mouse Brain

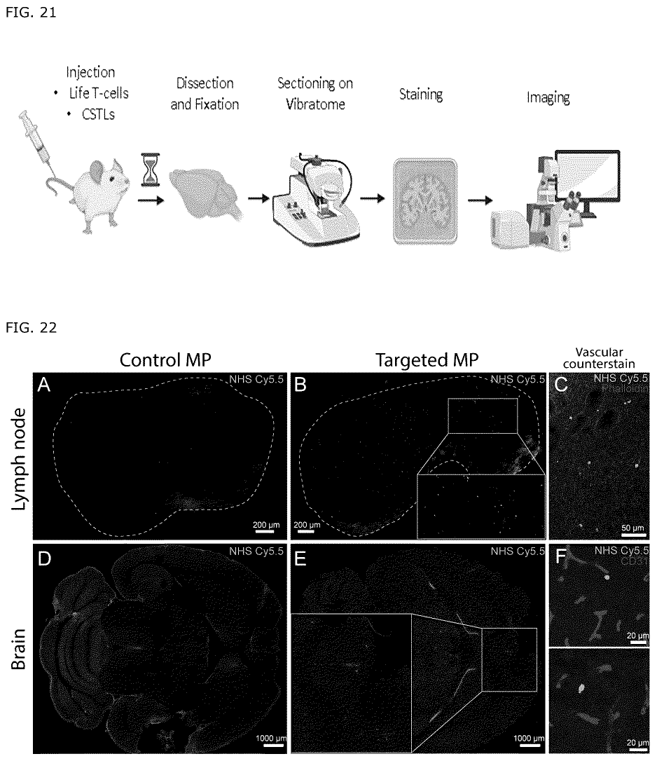

MPs crossing the blood-brain barrier and entry into brain parenchyma: Utilizing MPs to deliver therapeutic cargo into pharmacological sites of the body is an innovative and unexplored drug delivery system. The brain offers an interesting target site. To track the distribution of cell-mediated drug delivery vehicles in the brain and across the Blood-Brain Barrier (BBB), a mouse model was used. Experiments were performed using labeled syngeneically transferred live cells and compared to engineered syngeneic MPs (FIG. 21).

Preparation of live T-Lymphocyte injection: The injection that contains life T-cells was prepared right before the start of the experiment. The spleen was removed from healthy CD1 mice. The spleens were mechanically and enzymatically digested. Once the cell suspension without red blood cells was obtained, a magnetic antibody-binding bead system was used to isolate the T-Lymphocytes from other splenocytes. The pure T-Lymphocytes were incubated with a fluorescent CSFE solution. The cell suspension was injected intravenously into the lateral tail-vein of another healthy mouse.

Preparation of MP injection: To prepare the MP injection, live T-cells were obtained from a murine spleen like previously described. In order to produce MPs with a brain targeting phenotype, the isolated T cell pool was activated in vitro prior to cryopermeabilization. To induce generalized activation, isolated cells were incubated with 25 ng/ml Phorbol myristate acetate (PMA) and 1 ug/ml ionomycin for 6 hours to bypass the T cell membrane receptor complex and induce downstream membrane receptor alterations. After cryoinjury, these MPs were thawed and labeled with Cy5 as previously described for administration.

Imaging the brain: After different timepoints (4 h, 6 h, 8 h) the mouse was sacrificed following IACUC guidelines. After washing the brain it was fixed in 4% PFA with 0.1% Triton X-100 for 8-12 hours at 4° C. The tissue samples were embedded in 6% agarose gel and a vibratome was used to cut 300 micron thick sections. The tissue was blocked for 12 hours at 4° C. on the rocker followed by incubation for 48 hours at 4° C. on the rocker with rat-PECAM1 and rat-Endomucin to label the endothelial cells in the vasculature. The primary antibody was followed by three washes with 1×PBS for 5 minutes each followed by goat anti-rat Alexa Fluor 488 labeling.

Conclusions: Following administration of live T cells, untreated MPs, and activated MPs, it is clear that MP membrane phenotype plays an important role in tissue targeting (FIG. 21). There is significant evidence that activation of T cells is a crucial step for ingress into CNS tissues. In vitro “programming” of live cells with an activation cocktail to change membrane composition prior to creating MPs has been demonstrated as a viable strategy to produce targeted MPs. Phenotypic changes to cells were observed prior to MP production including increases of cell size, cellular extensions, and increased cell-cell aggregation. From administration in animals and confocal microscopy imaging, it is clear that activated MPs result in a complete change to targeting profile as LN uptake is reduced and brain uptake is greatly increased. Finally, as in the lymph node, vascular counterstaining shows escape of MPs from vessels and transport into brain parenchyma.

### EXAMPLE 6

### Initial PK/PD Modeling of MPs

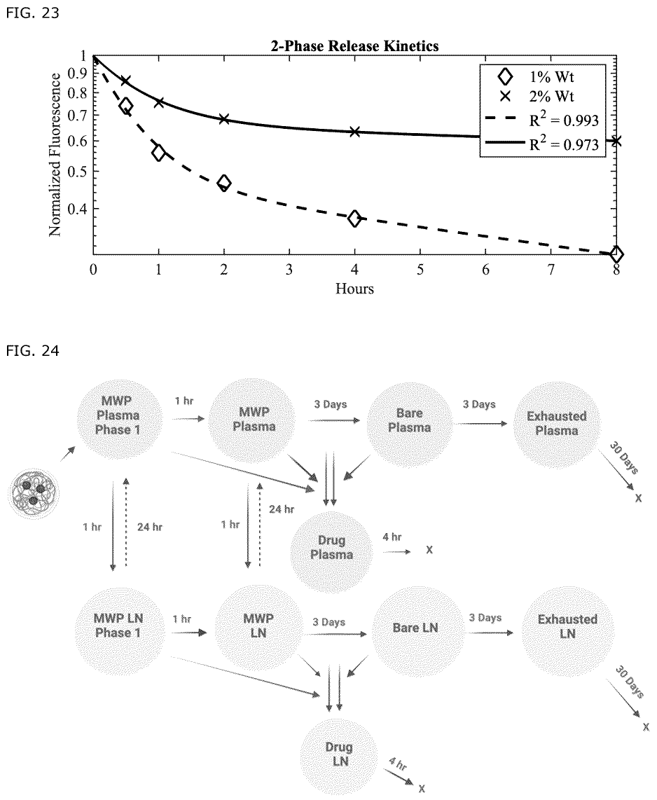

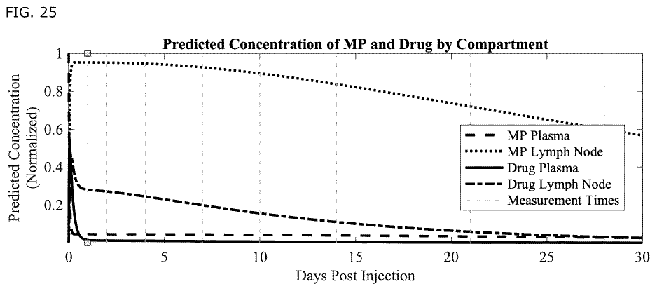

The microparticles have kinetics similar to living T cells, which are taken up from the blood into target tissues with an average residence time in the blood of 1 hour, and an average tissue residence time of approximately 24 hours. However, unlike living T cells, the microparticles usually cannot exit the tissues. Once the MPs enter the tissues, they remain in the tissues. We assume the membranes disassociate from the particles over 3-5 days, and the hydrogel cores slowly disintegrate over approximately 30 days, continually releasing their payload into the tissue. Our studies of small-molecule fluorophores has shown 2-phase release kinetics from the cores, with longer second-phase ½-lives for denser gel formulations (FIG. 23). The first-phase release kinetics have a ½-life on the order of 1 hour, and the second-phase release kinetics have a ½-life of approximately 4 days. We simulated the conditions that after all the drugs are released from the core, the exhausted particle remains in the tissue for approximately 30 days before disintegrating. These kinetics were captured in the PK/PD model (FIG. 24), and the predicted concentrations of the particles and a delivered drug are shown in FIG. 25. These results show that the particles should be able to achieve tissue-specific sustained release of payloads for several weeks.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for transporting a microparticle, wherein the microparticle comprises a core and a membrane surrounding the core, wherein the membrane comprises a cell membrane component, the method comprising:
(a) administering the microparticle to an endothelium, whereby the microparticle is bound to the endothelium; and
(b) moving the microparticle across the endothelium.

2. The method of claim 1, wherein the endothelium is in brain or a lymph node.

3. The method of claim wherein the endothelium is in a subject.

4. The method of claim 1, wherein the membrane further comprises a targeting moiety.

5. The method of claim 4, wherein the targeting moiety comprises an integrin, selectin, cadherin, immunoglobulin-like adhesion molecule, addressin, chemokine receptor, chemokine ligand, growth factor receptor, immunoglobulin superfamily protein, ion channel-linked receptor, G protein-coupled receptor, enzyme-linked receptor, antibody or a fragment thereof, or a binding domain thereof.

6. The method of claim 1, further comprising moving the microparticle to a target site after moving the microparticle across the endothelium.

7. The method of claim 6, wherein the endothelium is in a lymph node and the target site is a lobule in the lymph node.

8. The method of claim 6, wherein the endothelium is in a brain and the target site is in brain parenchyma or cerebrospinal fluid (CSF).

9. The method of claim 6, wherein the endothelium and the target site are in a tumor.

10. The method of claim 6, wherein the core comprises an active agent, further comprising releasing the active agent at the target site.

11. The method of claim 6, further comprising sequestering a molecule by the microparticle from the target site.

12. The method of claim 6, further comprising causing a biological response at the target site.

13. The method of claim 12, wherein the biological response is selected from the group consisting of immune interactions, cancer therapy, vaccine responses, and immunotherapy.

14. A microparticle comprising a core and a membrane surrounding the core, wherein the membrane comprises a cell membrane component.

15. The microparticle of claim 14, wherein the membrane further comprises a synthetic membrane component.

16. The microparticle of claim 14, wherein the membrane is from a permeabilized cell.

17. The microparticle of claim 16, wherein the permeabilized cell has been subject to cryopermeabilization, a detergent, or a chemical permeabilization solution.

18-20. (canceled)

21. The microparticle of claim 14, wherein the core comprises cytoplasm, a liquid, a polymer, an extracellular matrix protein, or a combination thereof.

22-25. (canceled)

26. The microparticle of claim 14, wherein the core is prepared from a leukocyte.

27. (canceled)

28. A method for preparing a microparticle, comprising mixing a core with a membrane, wherein the membrane comprises a cell membrane component.

29-40. (canceled)
